(19) United States
(12) Patent Application Publication
(10) Pub. No.: US 2024/0157149 A1
(43) Pub. Date: May 16, 2024

(54) SYSTEM AND METHOD FOR DEEP BRAIN STIMULATION

(71) Applicant: THE BOARD OF REGENTS OF THE UNIVERSITY OF TEXAS SYSTEM, Austin, TX (US)

(72) Inventors: Xiaoliang Wang, Dallas, TX (US); Bradley Lega, Dallas, TX (US)

(73) Assignee: THE BOARD OF REGENTS OF THE UNIVERSITY OF TEXAS SYSTEM, Austin, TX (US)

(21) Appl. No.: 18/502,619

(22) Filed: Nov. 6, 2023

Related U.S. Application Data
(60) Provisional application No. 63/383,127, filed on Nov. 10, 2022.

(51) Int. Cl. A61N 1/36; A61N 1/02; A61N 1/05; G16H 20/30

(52) U.S. Cl. CPC A61N 1/36135; A61N 1/025; A61N 1/0534; G16H 20/30

(57) ABSTRACT

A cystoscopy system with intracranial electrodes operable to be disposed in a hippocampus region of a brain and configured to record electrical signals that include biomarkers related to memory encoding, a neurostimulator configured to stimulate a posterior cingulate cortex (PCC) of the brain, a NARXNN plant model, and a controller configured to receive the electrical signals and modulate an input/output (I/O) relationship between the biomarkers and electrical stimuli applied to a posterior cingulate cortex (PCC) of the brain by controlling the neurostimulator to stimulate the PCC based on the I/O relationship to achieve a desired level of the biomarkers.

### CROSS-REFERENCE TO RELATED APPLICATION

This application claims priority to U.S. Provisional Patent Application Ser. No. 63/383,127, filed Nov. 10, 2022, and titled “SYSTEM AND METHOD FOR DEEP BRAIN STIMULATION,” which is incorporated by reference herein in its entirety.

### BACKGROUND

### 1. Field

The present application relates generally to deep brain stimulation and, more particularly, to a system and method of closed-loop control of deep brain stimulation.

### 2. Discussion of Related Art

Deep brain stimulation (DBS) with neuromodulation technique is an effective treatment for neurodegenerative disorders such as epilepsy, Parkinson's disease, post-traumatic amnesia, Alzheimer's disease, and neuropsychiatric disorders, such as, depression, obsessive-compulsive disorder, and schizophrenia. Typically, the neuromodulation strategies for neuromotor disorders utilize open-loop control, which relies solely on preset empirically derived stimulation parameters from clinical trials. However, unlike movement disorders, the underlying brain circuitry for memory disorders is more sophisticated and requires investigation of brain activity patterns or biomarkers as neuro-feedback to govern subsequent stimulation parameters. Open-loop schemes fail to capture the drastically changing dynamics of the neurological activities associated with cognitive processes, and some have been found to actually impair memory performance. In contrast, closed-loop stimulation systems that use data-driven models, such as, machine learning classifier triggered stimulation systems that deliver stimuli when the classifier predicts a memory failure. However, these systems are not accurate in modeling the input-output dynamics between posterior cingulate cortex (PCC) applied stimulation and stimuli-evoked hippocampal theta and gamma power. These systems also lack effectiveness in modulating theta and gamma power to a desired target level. It is with these observations in mind, among others, that various aspects of the present disclosure were conceived and developed.

### BRIEF SUMMARY

The present inventive concept addresses the foregoing problems by providing a method and system for deep brain stimulation. In one implementation, a method for deep brain stimulation includes recording, via intracranial electrodes, electrical signals in a hippocampus region of a brain, the electrical signals including biomarkers related to memory encoding; receiving, via a controller, the electrical signals including the biomarkers; modulating, via the controller, an input/output (I/O) relationship between the biomarkers and electrical stimuli applied to a posterior cingulate cortex (PCC) of the brain; and stimulating, via the controller and a neurostimulator, the PCC based on the I/O relationship to achieve a desired level of the biomarkers.

In another implementation, a system for deep brain stimulation is described and claimed herein. The system includes intracranial electrodes operable to be disposed in a hippocampus region of a brain and configured to record electrical signals that include biomarkers related to memory encoding; a neurostimulator configured to stimulate a posterior cingulate cortex (PCC) of the brain; and a controller configured to receive the electrical signals and modulating an input/output (I/O) relationship between the biomarkers and electrical stimuli applied to a posterior cingulate cortex (PCC) of the brain by controlling the neurostimulator to stimulate the PCC based on the I/O relationship to achieve a desired level of the biomarkers.

Additional aspects, advantages, and utilities of the present inventive concept will be set forth, in part, in the description which follows and, in part, will be obvious from the description, or may be learned by practice of the present inventive concept.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1 shows an example system for deep brain stimulation.

FIG. 2 shows an example proportional integral derivative (PID) controller of the system of FIG. 1.

FIG. 3 shows an example nonlinear autoregressive with exogenous input neural network (NARXNN) plant for training the controller of FIG. 1.

FIG. 4 shows a multilayer perceptron (MLP) neural network for use with the plant of FIG. 3.

FIG. 5 shows an example flow chart for using the system of FIG. 1 for deep brain stimulation.

FIG. 6 shows an illustration of batching input/output data into segments using temporal-wise attention (TA) techniques.

FIG. 7 shows an illustration of RMS gamma power when stimuli were applied to test subjects.

FIG. 8 shows an illustration of RMS theta power when stimuli were applied to test subjects.

FIG. 9 shows an illustration of overall performance for the system of FIG. 1 using one step ahead prediction where the controller is trained using a NARXNN plant versus a LSSM plant.

FIG. 10 shows an illustration of averaged normalized mean squared error in one-step-ahead prediction of RMS gamma and theta power for the system of FIG. 1 where the controller is trained using a NARXNN plant versus a LSSM plant.

FIG. 11 shows an illustration of a trace of full input driven predicted instantaneous theta and gamma RMS power for the system of FIG. 1 where the controller is trained using a NARXNN plant versus a LSSM plant.

FIG. 12 shows an illustration of averaged normalized mean squared error in full input driven prediction of theta and gamma RMS power for the system of FIG. 1 where the controller is trained using a NARXNN plant versus a LSSM plant.

FIG. 13 shows an illustration of power trials from closed-loop versus open-loop control using the system of FIG. 1.

FIG. 14 shown an illustration of power increases in simulated control of hippocampal theta and gamma power using the controller of FIG. 1 trained using a NARXNN plant versus a LSSM plant.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The illustrations and description are intended to describe aspects and embodiments of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps, or acts are in some way inherently mutually exclusive.

### II. General Architecture

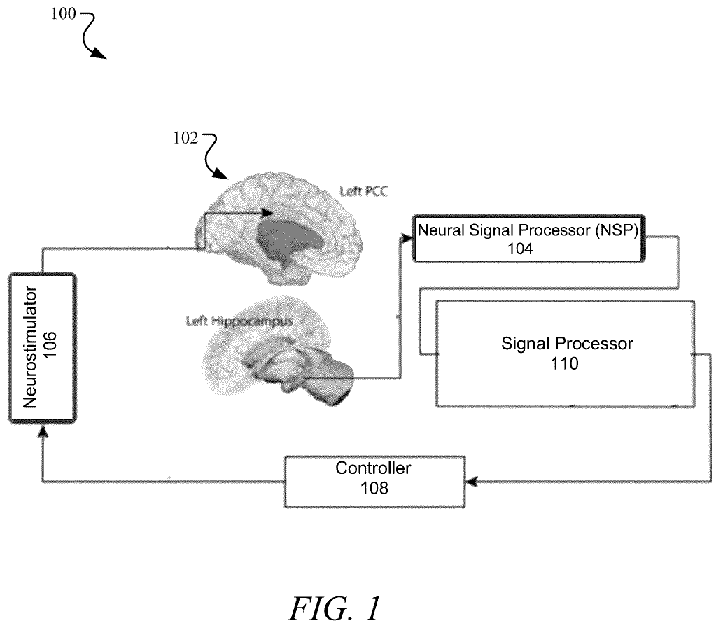

To begin a detailed description of an example system 100, reference is made to FIGS. 1-5. In one implementation, the system 100 includes intracranial electrodes disposed in a hippocampus region of a brain 102 and configured to record electrical signals using a neural signal processor (NSP) 104. The system 100 further includes a neurostimulator 106 configured to stimulate a posterior cingulate cortex (PCC) of the brain 102, and a controller 108 configured to receive the electrical signals and modulate an input/output (I/O) relationship between the biomarkers and electrical stimuli applied to a posterior cingulate cortex (PCC) of the brain 102 by controlling the neurostimulator 106 to stimulate the PCC based on the I/O relationship to achieve a desired level of the biomarkers. In an implementation, the electrical signals are denoised and processed by a signal processor 110 to extract the biomarkers before being received by the controller 108.

The NSP 104 utilizes intracranial electrodes disposed in a hippocampus region of a brain 102 to record electrical signals therein. In an implementation the electrical signals are acquired using intracranial electroencephalogram (iEEG). The electrical signals include biomarkers, such as, hippocampal theta and gamma oscillatory power, which are related to memory encoding.

The neurostimulator 106 is controlled via the controller 108 to stimulate the PCC of the brain 102 to modulate the changes in hippocampal oscillatory power. In an implementation, the neurostimulator 106 stimulates the PCC using binary-noise (BN) stimulation. Stimulating the posterior cingulate cortex (PCC) results in increases in hippocampal gamma power and conveys dynamical changes in the theta band.

The controller 108 receives the electrical signals recorded by the NSP 104 and modulates an input/output (I/O) relationship between the biomarkers and electrical stimuli applied to a posterior cingulate cortex (PCC) of the brain 102 by controlling the neurostimulator 106 to stimulate the PCC based on the I/O relationship to achieve a desired level of the biomarkers. The desired level of the biomarkers depends on certain memory-relevant biomarkers. A 50%-100% power increase, compared to the baseline, is considered desirable. For instance, this desired level could be between 80 to 120 microvolts for typical theta oscillations and 7 to 12 microvolts for typical gamma oscillations.

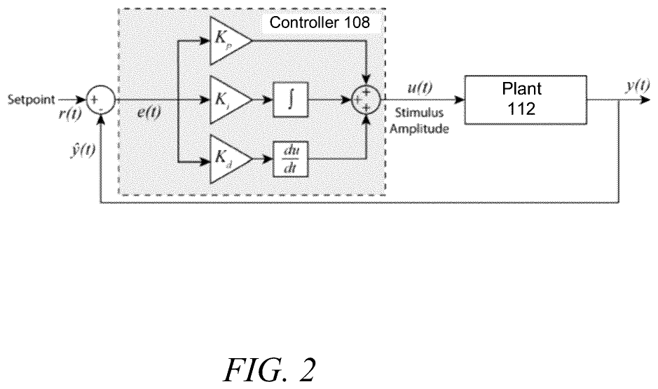

In an implementation, the controller 108 is a non-model-based control mechanism proportional integral derivative (PID) controller, as illustrated in FIG. 2. In this implementation, the controller 108 is tuned for a plant 112. In this implementation the controller 108 adjusts the control signal by referencing the error e(t) between setpoint and feedback. The control function of the controller 108 is given by:

$$u ( t ) = {K}_{p} e ( t ) + {K}_{i} {\int }_{0}^{t} e ( τ ) d ( τ ) + {K}_{d} \frac{de ( t )}{dt}$$

where Kₚ, Kᵢ, and K_{d} denote the gains for proportional, integral, and derivative terms, respectively.

In an implementation, the controller 108 is tuned to the plant 112 using the Ziegler-Nichols tuning method, where Kᵢ=Kₚ/Tᵢ and K_{d}=KₚT_{d}, and where Tᵢ and T_{d} are the integral and derivative time intervals, respectively. Thus, the control function of the controller 108 in this implementation is given by:

$$u ( t ) = {K}_{p} ( e ( t ) + \frac{1}{{T}_{i}} {\int }_{0}^{t} e ( τ ) d ( τ ) + {T}_{d} \frac{de ( t )}{dt} )$$

which has a transfer function of:

$$u ( s ) = {K}_{p} ( 1 + \frac{1}{{T}_{i} s} + {T}_{d} s ) e ( s ) = {K}_{p} ( \frac{{T}_{d} {T}_{i} {s}^{2} + {T}_{i} s + 1}{{T}_{i} s} ) e ( s )$$

The Ziegler-Nichols' first sets Kᵢ, and K_{d} to zero and increases Kₚ from zero until it reaches the ultimate gain Kᵤ at which the system outputs a stable and consistent oscillation, which has a period of Tᵤ. Then, it adjusts the Tᵢ and T_{d} based on the oscillation period Tᵤ. Ziegler and Nichols gave a typical rule for solving the PID parameter once ultimate gain is achieved as illustrated in Table 1 below.

| Control Type | Kₚ | Tᵢ | T_{d} | Kᵢ | K_{d} |
|---|---|---|---|---|---|
| P | 0.5Kᵤ | — | — | — | — |
| PI | 0.45Kᵤ | 0.80Tᵤ | — | 0.54Kᵤ/Tᵤ | — |
| PD | 0.8Kᵤ | — | 0.125Tᵤ. | — | 0.10KᵤTᵤ |
| PID | 0.6Kᵤ | 0.5Tᵤ | 0.125Tᵤ. | 1.2Kᵤ/Tᵤ | 0.075KᵤTᵤ |

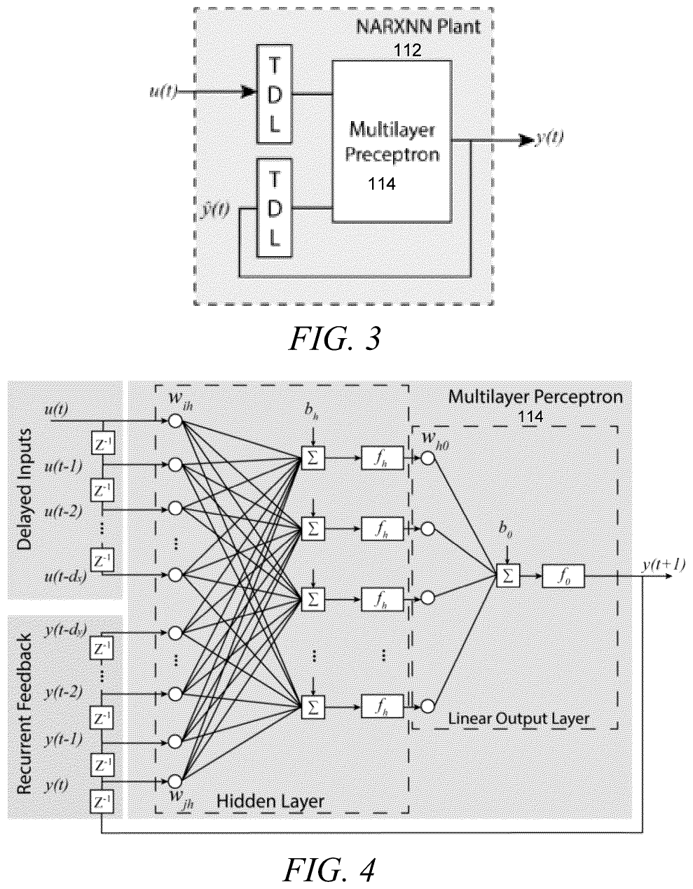

In an implementation the plant is a nonlinear autoregressive with exogenous input neural network (NARXNN), as illustrated in FIGS. 3 and 4. In an implementation, the NARXNN includes a linear autoregressive with exogenous input (ARX) model with nonlinear activation function optimized by a multilayer perceptron (MLP) neural network 114 arranged in a structure having less than three layers. In another implementation, the NARXNN is a two-layer NARXNN having a hidden layer and an output layer for modeling hippocampal theta and gamma oscillatory power, as illustrated in FIG. 4. In an implementation, the architecture of the NARXNN plant is represented as:

$$y ( t + 1 ) = {f}_{0} [ {b}_{0} + {\sum }_{h = 1}^{Nh} {w}_{h 0} \cdot {f}_{h} ( {b}_{h} + {\sum }_{i = 0}^{{d}_{u}} {w}_{ih} u ( t - 1 ) + {\sum }_{j = 0}^{{d}_{y}} {w}_{jh} y ( t - j ) ) ]$$

where wᵢₕ and wⱼₕ are the weights in the hidden layer for delayed input and output, respectively, bₕ is the bias in the hidden layer, wₕ₀ and b₀ are the weights and bias for the output layer, and f₀ (linear) and fₕ (sigmoid) are the activation functions of the output and hidden layer, respectively.

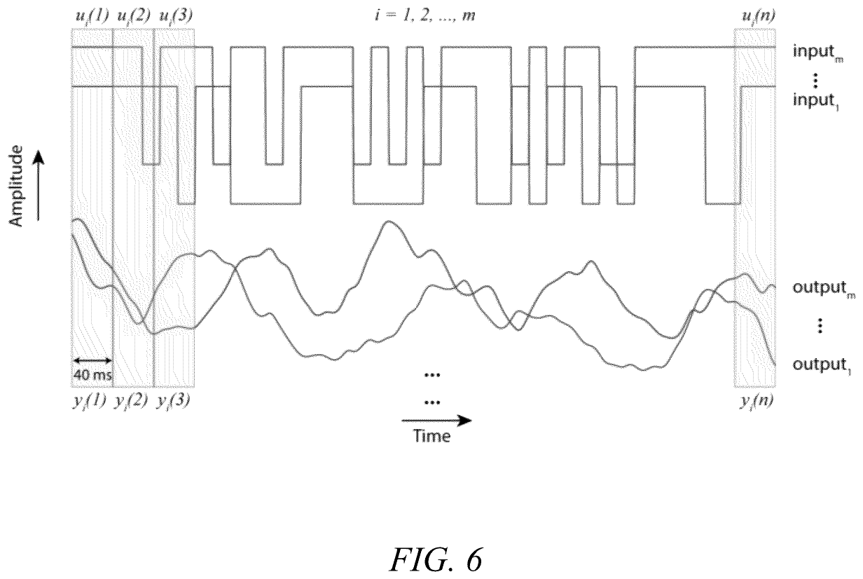

Referring to FIG. 6, in an implementation, the plant 112 is fitted to the I/O data using multi-sequence training and temporal-wise attention (TA) techniques. The multi-sequence training is a network training procedure used when the time-series data is not available in one long sequence. Thus, each epoch back-propagates and optimizes the network parameters on a trial-to-trial basis by concatenating all the I/O trials, and the delay is reset for each trial to maintain the same temporal scheme (i.e. yᵢ(t), . . . , yᵢ(t d_{y}) and uᵢ(t), . . . , uᵢ(t dᵤ), i=1, 2, m. As illustrated in FIG. 6, the I/O trials are batched input TA segments. On discrete sample basis, the input is now uᵢ=[uᵢ(1), uᵢ(2), . . . , uᵢ(n)] where i=1, 2, 3, . . . , m is the number of trials for the subject, and the output is yᵢ=[yᵢ(1), yᵢ(2), . . . , yᵢ(n)], accordingly. Each batched sample consists of a 20-sample long feature (40 ms) such that uᵢ(j)=[Iᵢ((j−1)×20+1), Iᵢ((j−1)×20+2), . . . , Iᵢ(j×20)] and yᵢ(j)=[Oᵢ((j−1)×20+1), Oᵢ((j−1)×20+2), . . . , Oᵢ(j×20)], where Iᵢ and Oᵢ are the original I/O trials. In this implementation, a Bayesian regularization algorithm to minimize the squared error. The algorithm uses the Levenberg-Marquardt backpropagation, which computes the Jacobian matrix jw of the mean squared error (MSE) with respect to the weight and bias variables w. Each variable is adjusted according to:

jj=jw^{T}jw

je=jw^{T}E

dw=−(jj+Iμ)je

where E is the matrix of errors, I is the identity matrix, and the step size μ is adaptively increased until the epoch reduces the performance in minimizing the MSE. The Bayesian regularization prevents overfitting and to smooth the network response. The parameters of a network are considered as random variables and can be written as:

$$P 〈 w \vert D \vert α , β , M 〉 = \frac{P ( D \vert w , β , M ) P ( D \vert w , α , M )}{P ( D \vert α , β , M )}$$

where α and β are parameters for the objective function such that E=βE_{D}+αE_{W}, where E_{W} is the sum of squares of the network weights and E_{D} is sum of squared errors E_{D}=Σⱼ₌₁ⁿ(y(j)−ŷ(j))². D is the data set, M is the network model used, and w is the vector of network parameters. P (w|α, M) is the prior density representing the knowledge of the network parameter prior to any data collection. P (D|w, β, M) is the likelihood of the data occurring, given the parameter w. P (D|α, β, M) is a normalization factor with total probability of 1. The normalization factor can be solved by:

$$P ( D \vert α , β , M ) = \frac{P ( D \vert w , β , M ) P ( w \vert α , M )}{P 〈 w \vert D \vert α , β , M 〉} = \frac{[ \frac{1}{{Z}_{D} ( β )} exp ( - β {E}_{D} ) ] [ \frac{1}{{Z}_{W} α} esp ( - α {E}_{W} ) ]}{\frac{1}{{Z}_{F} ( α , β )} exp ( - F ( w ) )}$$

$$\frac{{Z}_{F} ( α , β )}{{Z}_{D} ( β ) {Z}_{W} ( α )} \cdot \frac{exp ( - β {E}_{D} - α {E}_{W} )}{exp ( - F ( w ) )}$$

$$\frac{{Z}_{F} ( α , β )}{{Z}_{D} ( β ) {Z}_{W} ( α )}$$

$$and$$

$${Z}_{F} ( α , β ) \approx {( 2 π )}^{\frac{N}{2}} {( det ( {( {H}^{MP} )}^{- 1} ) )}^{\frac{1}{2}} exp ( - F ( {w}^{MP} ) )$$

$${Z}_{D} ( β ) = \frac{1}{P ( D \vert W , β , M )} exp ( - β {E}_{D} )$$

$${Z}_{W} ( α ) = \frac{1}{P ( w \vert α , M )} exp ( - α {E}_{W} )$$

where H=β∇²E_{D}+α∇²E_{W} is the Hessian matrix of the objective function. The Bayesian regularization minimizes the linear combination of squared errors and weights so that the resulting network has good generalization qualities at the end of training.

In an implementation, the signal processor 110 is a set of algorithms for noise removal and biomarker extraction that includes an outlier removal algorithm based on the mean Euclidean distance (MED) method, a denoising algorithm via a signal-subspace approach, a finite impulse response (FIR) notch filter for 60 Hz line noise removal, an FIR bandpass filter for theta and/or gamma oscillation(s) extraction, and a power extraction algorithm using the Hilbert transform. In an implementation, the signal processor 110 is operable to denoise the electrical signals recorded by the NSP 104 prior to the electrical signals being received by the controller 108. In this implementation, mean Euclidean distance (MED) is computed for each trial with respect to a reference trial. The MED is represented by:

$$M E D ( i ) = \sqrt{\frac{1}{N} {\sum }_{j = 1}^{n} {( {x}_{i} ( j ) - {x}_{τ} ( j ) )}^{2}}$$

where i=1, 2, 3 . . . , n is the trials number, j=1, 2, 3 . . . , N is the sample number of the trial, and x_{T} is the reference trial. The outliers that exceed a range from the reference trial can be rejected. For instance, trials that exhibit 75% greater than the MED of the reference trial are rejected. However, the disclosure is not limited as such, and any suitable range can be used. The remaining trials can down sampled, such as, for example, to 500 Hz, followed by an anti-aliasing lowpass filtering, such as, for example, at 200 Hz, and further denoised via a subspace approach represented by:

B=AQₛQₛ^{T}=[s₁(t)^{T},s₂(t)^{T},s₃(t)^{T}, . . . , sₘ(t)^{T}]^{T}

where A=[x₁(t)^{T}, x₂(t)^{T}, x₃(t)^{T}, . . . , xₘ(t)^{T}]^{T} is the matrix of the trials, Qₛ is the matrix of the principle eigenvectors decomposed from the sample correlation matrix \hat{R}=A^{T}A, and sᵢ, I=1, 2, 3, . . . , M are the projections of the trials onto the signal subspace.

In an implementation, the signal processor 110 is further operable to extract the biomarkers from the electrical signals recorded by the NSP 104 prior to the electrical signals being received by the controller 108. In an implementation, denoised iEEG signals are bandpass filtered via an FIR filter into theta and/or gamma oscillation(s), and the instantaneous root mean square (RMS) power is extracted from the theta and/or gamma oscillation(s) by applying the Hilbert transform, followed by taking the absolute value.

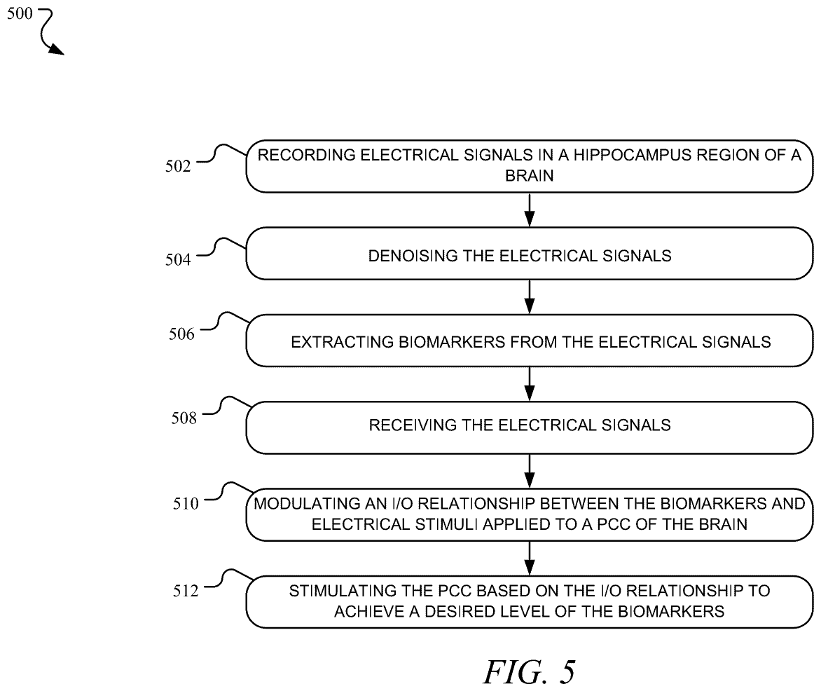

Referring to FIG. 5, a method 500 for performing deep brain stimulation via the system 100 is shown. The method 500 is provided by way of example, as there are a variety of ways to carry out the method. The method 500 described herein can be carried out using the configurations and examples illustrated in the figures, for example, and various elements of these figures are referenced in explaining the method 500. Each block shown in FIG. 5 represents one or more processes, methods, or subroutines, carried out in the method 500. Furthermore, the illustrated order of blocks is illustrative only and the order of the blocks can change according to the present disclosure. Additional blocks may be added, or fewer blocks may be utilized, without deviating from the scope of the present disclosure. The method 500 can begin at block 502.

At block 502, electrical signals are recorded via intracranial electrodes disposed in a hippocampus region of a brain using the NSP 104. The electrical signals including biomarkers related to memory encoding, such as, for example, hippocampal theta and gamma oscillatory power. In an embodiment, the electrical signals are acquired using intracranial electroencephalogram (iEEG).

At block 504, the electrical signals including the biomarkers are denoised by the signal processor 110. At block 506, the biomarkers are extracted from the electrical signals by the signal processor 110.

At block 508, the electrical signals are received by the controller 108. At block 510, the controller 108 modulates an input/output (I/O) relationship between the biomarkers and electrical stimuli applied to a posterior cingulate cortex (PCC) of the brain 102. In an implementation, the controller 108 is a proportional integral derivative (PID) controller tuned for the plant 112. In an implementation, the plant 112 is a nonlinear autoregressive with exogenous input neural network (NARXNN). In an implementation, the NARXNN includes a linear autoregressive with exogenous input (ARX) model with nonlinear activation function optimized by a multilayer perceptron (MLP) 114 neural network arranged in a structure having less than three layers, such as, for example a two-layer NARXNN having a hidden layer and an output layer for modeling hippocampal theta and gamma oscillatory power.

At block 512, the controller 108 controls the neurostimulator 106 to stimulate the PCC of the brain 102 based on the I/O relationship to achieve a desired level of the biomarkers.

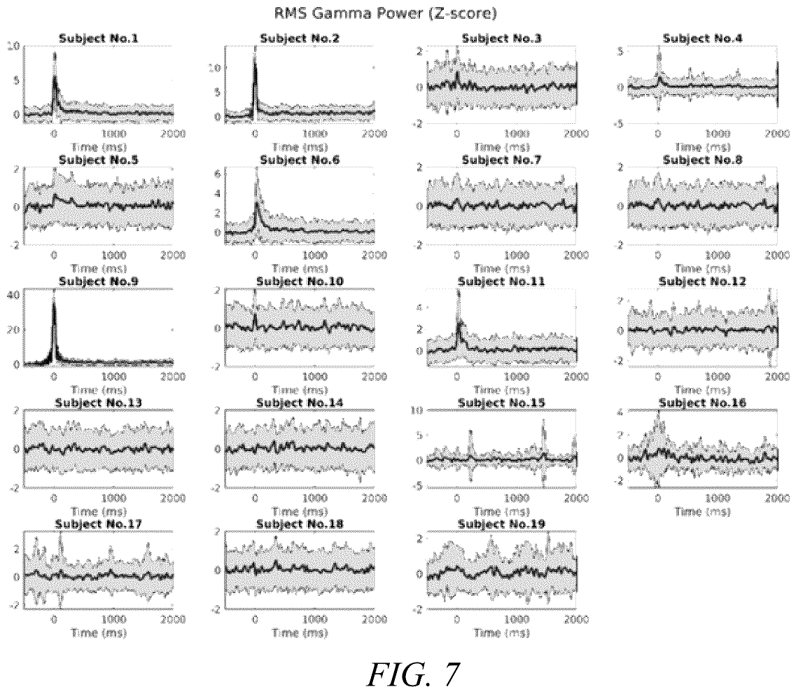

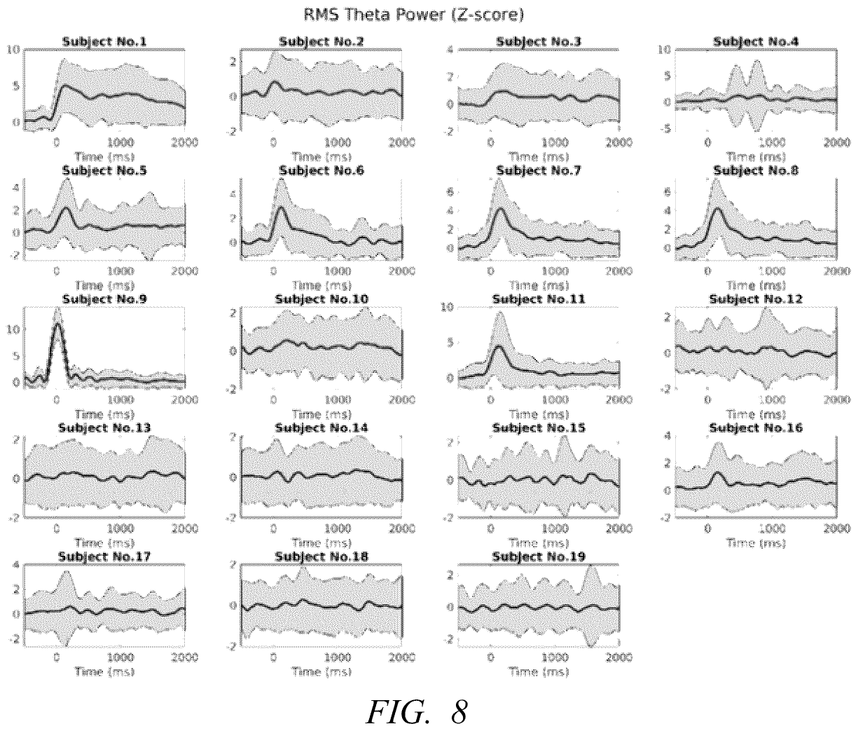

Method 500 implemented via system 100 has been found by the inventors to be more effective at modulating theta and gamma hippocampal power to improve episodic memory retention compared to conventional methods. During a study of 19 subjects, dynamical changes of the stimuli-evoked hippocampal oscillatory power in the theta and gamma bands were investigated. In the study, the theta and gamma RMS power trials in a time window of 500 ms before the stimulation (i.e., −500 ms) to 2000 ms after the stimulation (i.e., +2000 ms), where stimuli were onset at 0 ms. Because of the various power levels across different subjects, the power trials were normalized in z-scores by subtracting the mean then dividing by the standard deviation. Increases in the gamma power were observed within 49.36±21.68 ms after stimulation in 15 out of 19 subjects. Similar for the theta power, power increases were observed at an average of 89.15±55.32 ms in 15 of 19 subjects. These results are found in FIGS. 7-8.

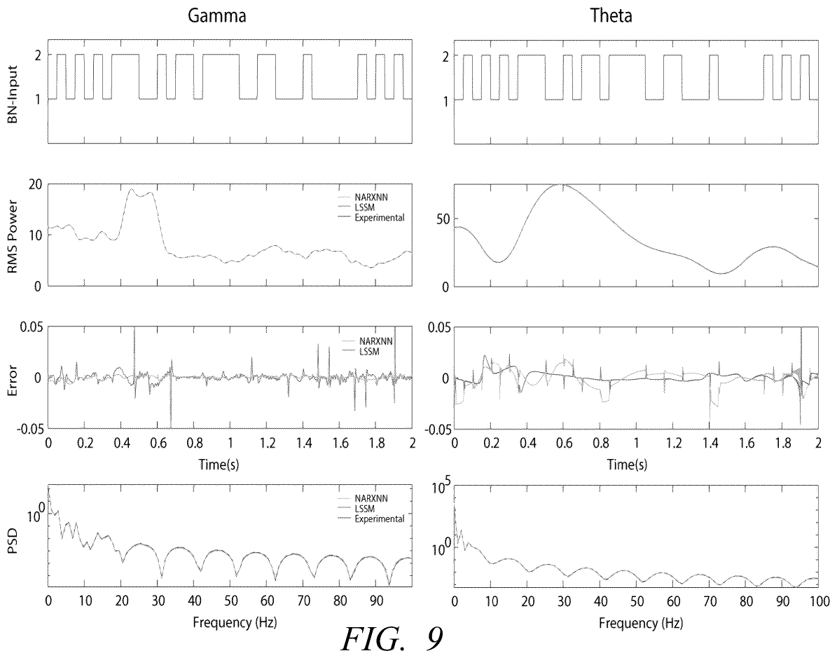

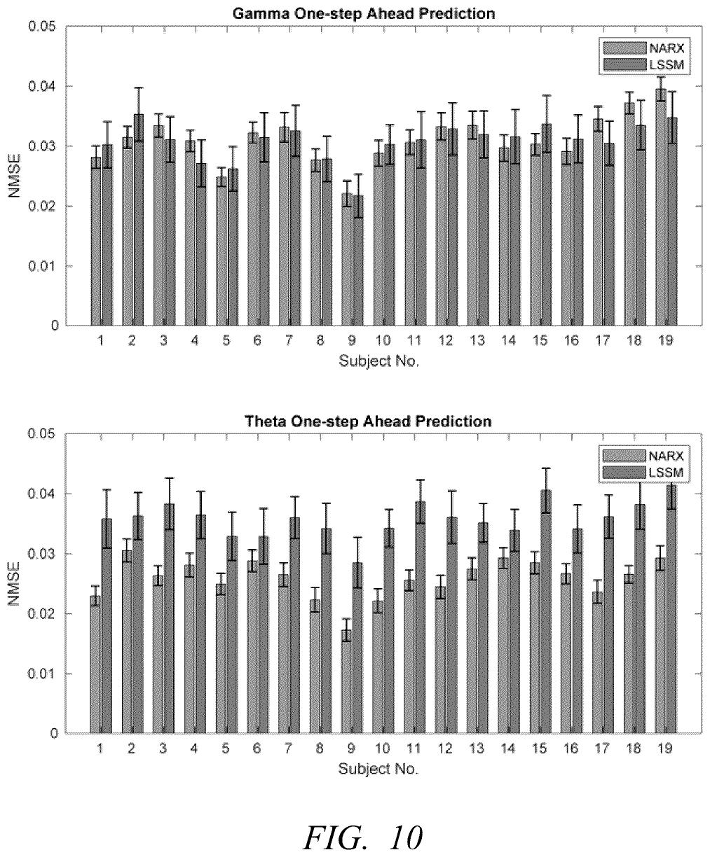

For the 19 subjects, the NARXNN plant was compared to a LSSM plant. Across 19 subjects, both the NARXNN and LSSM plants accurately predicted the input-driven dynamics of the hippocampal theta and gamma oscillatory activities in response to stimulation. One-step-ahead prediction was performed, where the predictor estimates the next output given the knowledge of current and previous input and output. Both the LSSM and NARXNN plants predicted instantaneous theta and gamma RMS power closely to experimental trials, as expected. FIG. 9 shows an example trace of predicted instantaneous theta and gamma RMS power via LSSM and NARXNN plants, where the prediction error and the power spectral density (PSD) of the predictions versus the experimental data are compared. The overall performance for subject-specific NARXNN versus LSSM in one-step ahead prediction is shown in FIG. 10. Across all subjects, the averaged NMSE of NARXNN predictions was 0.0342±0.007 for the gamma power and 0.0269±0.0010 for the theta power, and the averaged NMSE of LSSM predictions was 0.0322±0.006 for the gamma power and 0.0389±0.008 for theta power.

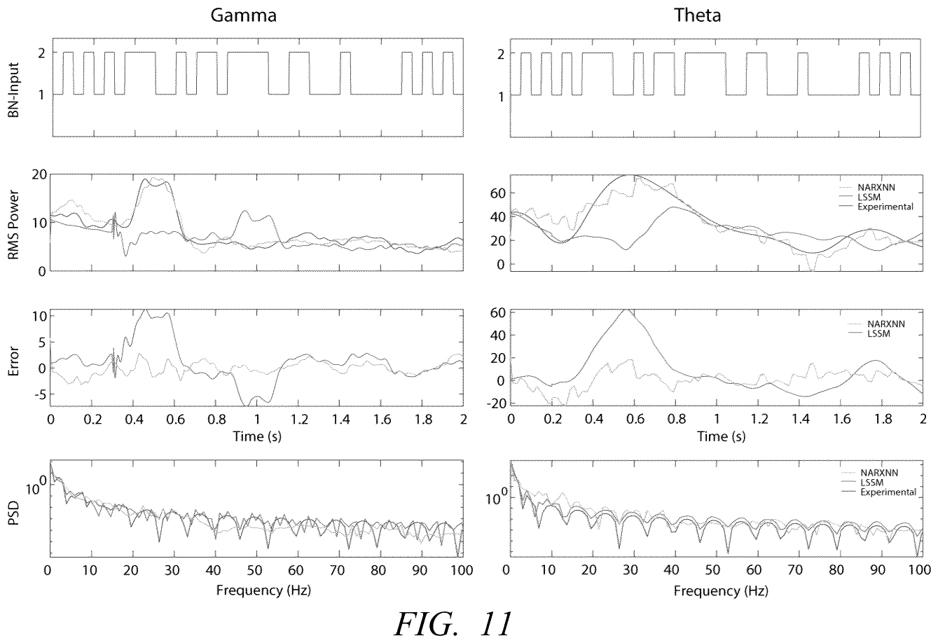

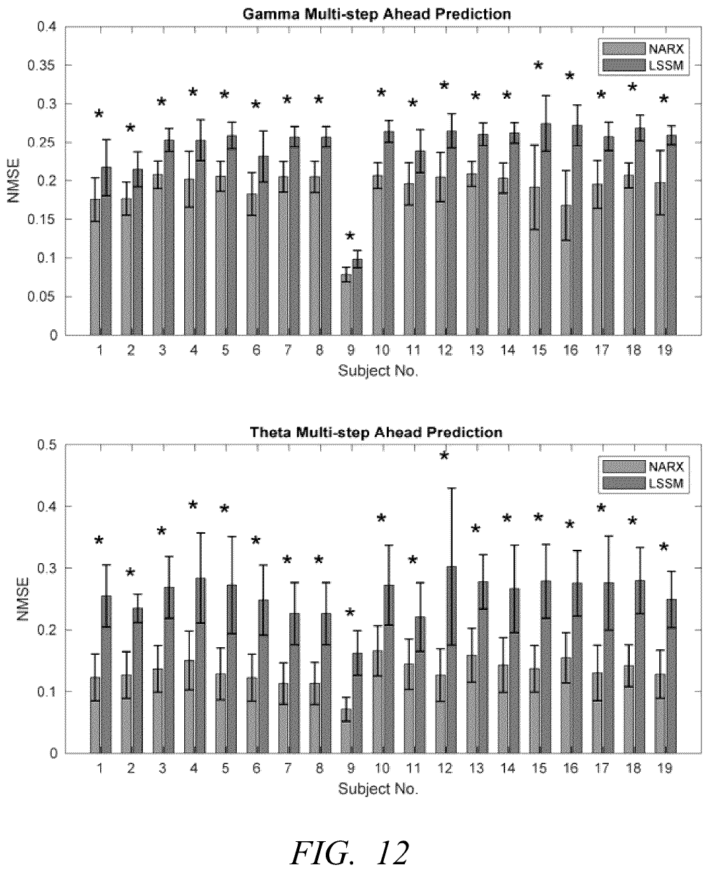

Full input-driven prediction in both models was implemented. Here, the plants are “simulators” (simulation focused) instead of “predictors” (prediction focused). This input-driven prediction represents how a plant responses to a system input and yields input-driven dynamics of the system. As shown in FIG. 11, the example trace shows that NARXNN introduced some random fluctuation in predicting gamma and theta power but maintained a fair match to the experimental trial, whereas LSSM lost critical temporal resolutions especially within the 0.3-0.8 s time window where theta and gamma exhibited great dynamical changes that LSSM failed to predict accurately. Across all subjects, the averaged NMSE of predictions via NARXNN was 0.179±0.055 for the gamma power and 0.0153±0.071 for the theta, and the averaged NMSE of predictions via LSSM was 0.259±0.082 for the gamma band and 0.288±0.079 for the theta. In all 19 subjects, gamma band power predicted by NARXNN model exhibited less NMSE comparing to the predictions by LSSM model, and they all had significant less NMSE via paired t-tests (p<0.05). For theta power prediction, 19 subjects had less NMSE using NARXNN than using LSSM and all of the subjects were statistically significant (see FIG. 12). Thus, it is shown that the NARXNN plant outperforms the LSSM plant.

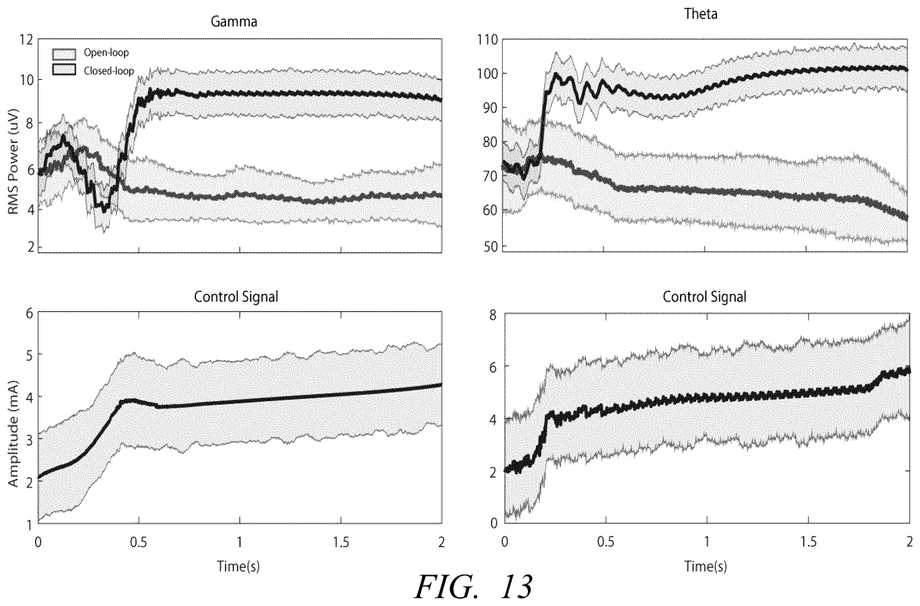

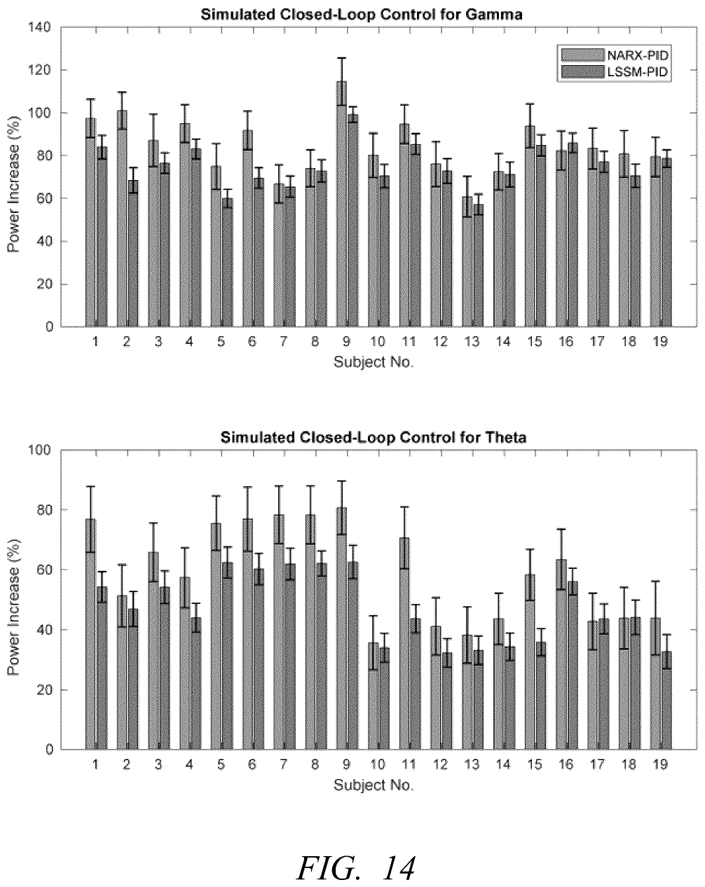

A simulated testbed for the system 100 was created and conducted closed-loop control was conducted using the NARXNN plant, paired with the controller 108 for testing the effectiveness and efficiency in modulating hippocampal theta and gamma power. The controller 108 for each subject-specific NARXNN plant was manually tuned following the Ziegler-Nichols' method, independently. Then, a subject-specific target power level for each theta and gamma oscillations was determined by the highest power level the system 100 was able to achieve while maintaining the control signal (i.e., stimulation amplitude) in a psychological safe range of 0-9 mA. Then, the closed-loop control took place over a course of 2 s, where closed-loop stimulation was onset at t=0, for 76.6±18.8 independent simulations across the 19 subjects. Open-loop stimulation was also simulated using the experimental stimulation input. Example trials of closed-loop control versus open-loop control are found in FIG. 13 where closed-loop control of gamma power was 87.3%±12.6% greater than open-loop power, and closed-loop control of theta power was 56.7%±13.3% greater, using the system 100. This was calculated on averaged theta/gamma power across the entire 2 s of simulation. The averaged time to achieve setpoint using the system 100 was 413.63 ms±211.32 ms for the gamma power and 186.36 ms±59.23 ms for the theta. For comparison, a LSSM-PID closed-loop control architecture was also tested. The LSSM-PID scheme was able to reach a target level, but the control signals were saturated at safety boundary and were not as physiologically realistic as the system 100, which gradually increased the stimulation amplitude to compensate the intrinsic descent hippocampal power to maintain the desired level (see control signals in FIG. 13). By selecting a proper setpoint for each subject, we compared the closed-loop control performance for both the system 100 and LSSM-PID framework. The capability of both systems in modulating and controlling oscillatory power was found and confirmed the superior performance using the architecture of system 100. In 17 of 19 subjects, closed-loop control using the system 100 exhibited greater gamma power increases versus LSSM-PID under the same safety guideline, and 13 of them were statistically significant (p<0.05). The averaged gamma power increase in the closed-loop control using the system 100 was 83.21%±17.62% versus 72.53%±1543.% using the LSSM-PID. For the theta power, we found that 17 out of 19 subjects had greater power increases using the system 100 than LSSM-PID (17 were significant, p<0.05). The averaged theta power increase in closed-loop control, comparing to the experimental power, was 60.05%±15.07% using the system 100 versus 46.67%±9.98% via LSSM-PID. These results are visible in FIG. 14. Thus, the system 100 is superior in modulating oscillatory power compared to the LSSM-PID framework.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall therebetween.

What is claimed is:

1. A method for deep brain stimulation, the method comprising:
recording, via intracranial electrodes, electrical signals in a hippocampus region of a brain, the electrical signals including biomarkers related to memory encoding;
receiving, via a controller tuned for a plant, the electrical signals including the biomarkers;
modulating, via the plant and the controller, an input/output (I/O) relationship between the biomarkers and electrical stimuli applied to a posterior cingulate cortex (PCC) of the brain; and
stimulating, via the controller and a neurostimulator, the PCC based on the I/O relationship to achieve a desired level of the biomarkers.

2. The method of claim 1, wherein the electrical signals are acquired using intracranial electroencephalogram (iEEG).

3. The method of claim 1, wherein the biomarkers include hippocampal theta and gamma oscillatory power.

4. The method of claim 1, wherein the recording is performed using a neural signal processor (NSP).

5. The method of claim 1, wherein the controller is a proportional integral derivative (PID) controller.

6. The method of claim 5, wherein the plant is a nonlinear autoregressive with exogenous input neural network (NARXNN).

7. The method of claim 6, wherein the NARXNN includes a linear autoregressive with exogenous input (ARX) model with nonlinear activation function optimized by a multilayer perceptron (MLP) neural network arranged in a structure having less than three layers.

8. The method of claim 6, wherein the NARXNN is a two-layer NARXNN having a hidden layer and an output layer for modeling hippocampal theta and gamma oscillatory power.

9. The method of claim 1 further comprising:
denoising the electrical signals; and
extracting the biomarkers from the electrical signals.

10. A system for deep brain stimulation, the system comprising:
intracranial electrodes operable to be disposed in a hippocampus region of a brain and configured to record electrical signals that include biomarkers related to memory encoding;
a neurostimulator configured to stimulate a posterior cingulate cortex (PCC) of the brain; and
a controller configured to receive the electrical signals and modulating an input/output (I/O) relationship between the biomarkers and electrical stimuli applied to a posterior cingulate cortex (PCC) of the brain by controlling the neurostimulator to stimulate the PCC based on the I/O relationship to achieve a desired level of the biomarkers.

11. The system of claim 10, wherein the electrical signals are acquired using intracranial electroencephalogram (iEEG).

12. The system of claim 10, wherein the biomarkers include hippocampal theta and gamma oscillatory power.

13. The system of claim 10, wherein the intracranial electrodes record the electrical signals using a neural signal processor (NSP).

14. The system of claim 10, wherein the controller is a proportional integral derivative (PID) controller.

15. The system of claim 14, wherein the PID controller is tuned for a plant.

16. The system of claim 15, wherein the plant is a nonlinear autoregressive with exogenous input neural network (NARXNN).

17. The system of claim 16, wherein the NARXNN includes a linear autoregressive with exogenous input (ARX) model with nonlinear activation function optimized by a multilayer perceptron (MLP) neural network arranged in a structure having less than three layers.

18. The system of claim 16, wherein the NARXNN is a two-layer NARXNN having a hidden layer and an output layer for modeling hippocampal theta and gamma oscillatory power.

19. The system of claim 10 wherein the electrical signals are denoised and processed by a signal processor to extract the biomarkers before being received by the controller.
